(19) United States
(12) Patent Application Publication
(10) Pub. No.: US 2007/0043247 A1
(43) Pub. Date: Feb. 22, 2007

(54) Diisobutylene production

(76) Inventors: Kenneth M. Webber, Friendswood, TX (US); Thomas S. Zak, West Chester, PA (US)

Correspondence Address: LYONDELL CHEMICAL COMPANY, 3801 WEST CHESTER PIKE, NEWTOWN SQUARE, PA, 19073, US

(21) Appl. No.: 11/207,272

(22) Filed: Aug. 19, 2005

(51) Int. Cl. C07C 2/04

(52) U.S. Cl. 585/510

(57) ABSTRACT

A method for forming diisobutylene from a hydrocarbon stream that contains acetylenics and a low concentration of isobutylene, comprising at least reducing the acetylenic content of the stream before catalytically oligomerizing the isobutylene to diisobutylene.

### BACKGROUND OF THE INVENTION

This invention relates to the formation of diisobutylene (isooctene) from hydrocarbon streams that contain isobutylene in low concentrations. In particular, this invention relates to the production of diisobutylene (DIB) from streams that predominantly contain a mixture of compounds having four carbon atoms per molecule (C₄'s), such as the C₄ streams that are generated in hydrocarbon cracking plants.

### DESCRIPTION OF THE PRIOR ART

Although this invention will, for sake of clarity and brevity, be described in respect of a C₄ mixture obtained from a hydrocarbon thermal cracking plant, this invention is not so limited. It can be applicable to C₄ streams of similar composition, however generated, or otherwise obtained.

Thermal cracking of hydrocarbons is a petrochemical process that is widely used to produce olefins such as ethylene, propylene, butenes, butadiene, and aromatics such as benzene, toluene, and xylenes. In an olefin production plant, a hydrocarbonaceous feedstock such as ethane, naphtha, gas oil, or other fractions of whole crude oil is mixed with steam which serves as a diluent to keep the hydrocarbon molecules separated. This mixture, after preheating, is subjected to severe hydrocarbon thermal cracking at elevated temperatures (1,450 to 1,550 degrees Fahrenheit, or F.) in a pyrolysis furnace (steam cracker or cracker).

The cracked product effluent of the pyrolysis furnace (furnace) contains hot, gaseous hydrocarbons of great variety (from 1 to 35 carbon atoms per molecule, or C₁ to C₃₅, inclusive). This product contains aliphatics (including alkanes and alkenes), alicyclics (including cyclanes, cyclenes and cyclodienes), aromatics, saturates, and unsaturates, and molecular hydrogen (hydrogen).

This furnace product is then subjected to further processing to produce, as products of the olefin plant, various, separate and individual product streams such as hydrogen, ethylene, and propylene. After the separation of these individual streams, the remaining cracked product contains essentially C₄ hydrocarbons and heavier. This remainder is fed to a debutanizer wherein a crude C₄ stream is separated as overhead while a C₅ and heavier stream is removed as a bottoms product.

Such a C₄ stream can contain varying amounts of n-butane, isobutane, 1-butene, 2-butenes (both cis and trans isomers), isobutylene, acetylenes, and diolefins such as butadiene (both 1,2 and 1,3 isomers). At least about 40 weight percent (wt. %) of this stream will be made up of a mixture of 1,3 butadiene and 1,2 butadiene. This stream can contain a significant but minor amount of mono-olefins (1-butene, 2-butenes and isobutylene), i.e., up to about 50 wt. %. All wt. % are based on the total weight of the stream.

Heretofore, this crude C₄ stream has typically been subjected to extractive distillation to remove diolefins, particularly 1,3 butadiene, from the C₄ stream, and produce a C₄ raffinate stream. See U.S. Pat. Nos. 3,436,438, and 4,134,795. The C₄ raffinate stream was then subjected to, for example, an etherification step to convert at least part of its isobutylene content to methyl t-butyl ether, or a metathesis step to convert at least part of its 2-butene content to propylene. This raffinate stream was not typically used to convert any of its isobutylene (C₄H₈) content to DIB (C₈H₁₆).

Heretofore, the prior art of converting isobutylene to DIB has employed only C₄ streams containing very high concentrations of isobutylene, e.g., at least about 95 wt. % isobutylene based on the total weight of the stream, to form DIB. See U.S. Pat. Nos. 5,877,372 and 6,376,731. This was due, in part, to the fact that when low concentration isobutylene streams such as the crude C₄ streams described above were used to produce DIB, an unexpectedly high rate of catalyst fouling was experienced.

DIB, and its corresponding saturate, isooctane, are useful as high octane blending components. Accordingly, it is desirable to be able to use C₄ streams that have a low concentration of isobutylene as a source of DIB.

### SUMMARY OF THE INVENTION

It has been found that the source of the problem in using low concentration isobutylene streams to form DIB lay in the acetylenic content of the stream.

Pursuant to this invention, mixed C₄ streams that contain low concentrations of isobutylene are employed as a source of DIB by first at least reducing, if not essentially removing, the acetylenics from the stream, followed by dimerization of at least part of the isobutylene in that stream to DIB.

Accordingly, by this invention, DIB sources are no longer limited to high concentration isobutylene streams; low concentration isobutylene streams as defined herein now being useful for the same purpose.

### BRIEF DESCRIPTION OF THE FIGURES

FIG. 1: Unused Catalyst Pellets

This figure is a magnified image of several pellets of a polymer based macroporous sulfonic acid ion exchange resin, showing the shape of pellets before being used in the reaction process.

FIG. 2: Catalyst Pellets Exposed to Feeds with Ethyl Acetylene

This figure is a magnified image of several catalyst pellets removed from a reactor in which the feed contained a measurable concentration of ethyl acetylene

FIG. 3: Catalyst Pellets Exposed to Feeds without Ethyl Acetylene

This figure is a magnified image of several catalyst pellets removed from a reactor in which the feed contained no detectable level of ethyl acetylene

### DETAILED DESCRIPTION OF THE INVENTION

DIB is normally present as a mixture of two isomers; 2,4,4-trimethyl-1-pentene and 2,4,4-trimethyl-2-pentene. This invention is applicable to either isomer, or a mixture of such isomers in any proportions, all of which are generically referred to herein as DIB.

The feed material for the process of this invention is deliberately chosen to be one that contains a minor amount of isobutylene, and some acetylenics, all as defined hereinafter.

A typical feed is a C₄ stream produced as the raffinate stream during the extraction of butadiene. Such feeds can contain a major amount, at least about 50 wt. % of isobutylene, and a significant, but minor, amount (up to but no more than about 50 wt. %, e.g., from about 30 to about 50 wt. %) of at least one of 1-butene and 2-butenes (butenes). The butenes can be present in varying amounts, e.g., from about 20 to about 30 wt. % 1-butene, and from about 10 to about 20 wt. % 2-butenes. This feed can also contain very minor amounts of n-butane (from about 3 to about 10 wt. %), isobutane (from about 1 to about 5 wt. %), and diolefins such as butadiene (less than about 2 wt. %, typically from about 0.5 to about 1 wt. %). All wt. % are based on the total weight of the C₄ stream. The compounds present in the least amount in such a feed stream (from about 100 to about 1,000 parts per million, or ppm) are the acetylenics, typically vinylacetylene and ethylacetylene.

The small amount of acetylenics present were not heretofore thought to be significant in respect of causing problems in the downstream processing of this type of stream. This is, in part, why it was a surprise to find that the acetylenics were the source of the problem in forming DIB from this type of low concentration isobutylene stream. However, such was found to be the case. As shown hereinafter, when acetylenics were removed from streams of this type of composition, catalyst fouling that was previously experienced when subjecting such a stream to isobutylene dimerizing conditions fell dramatically.

The catalysts and conditions for dimerizing isobutylene to DIB are well known. Generally, such conditions include a temperature of from about 150 to about 250° F., a pressure of from about 250 to about 400 psig, and a weight hourly space velocity of fresh feed from about 0.5 to about 10 reciprocal hours. Suitable, non-limiting, catalysts for this reaction include macroporous ion exchange resin made up of sulfonated polystyrene resins crosslinked with divinylbenzene.

Acetylenics can be at least reduced, and even essentially removed (at least down to non-detectable amounts), from the subject feed stream in a number of ways known in the art.

One such method is the selective hydrogenation of the acetylenics to the corresponding olefin. In this method the feed is mixed with from about 1 to about 10 moles of molecular hydrogen per mole of acetylenics at a temperature of from about 60 to about 150° F., a pressure of from about 100 to about 500 psig, and a weight hourly space velocity of from about 5 to about 10 reciprocal hours. Non-limiting suitable catalysts include palladium supported on alumina, platinum supported on alumina, sulfided nickel supported on alumina.

Another such method involves hydrogenation of the acetylenics and the oligomerization of the acetylenics to heavier compounds such as C₈ unsaturated moities. These heavier compounds can thereafter be separated from the stream by simple fractional distillation. These oligomerization methods are known in the art. For example, acetylenic containing crude C₄ streams can be subjected to conditions that favor acetylenic oligomerization that include a temperature of from about 75° F. to about 200° F., a pressure of from about 50 to about 500 psig, and a weight hourly space velocity of from about 2 to about 10 reciprocal hours. Suitable non-limiting catalysts include nickel modified copper oxide on an alumina support. The resulting acetylenic oligomers can be separated from the feed stream by distillation at a temperature of from about 100 to about 200° F., under a pressure of from about 50 to about 100 psig.

### EXAMPLE 1

A sample of a mixed C₄ stream (Raff1) was employed that had a composition of 39 weight percent (wt. %) isobutylene, 40 wt. % normal butenes, 0.86 wt. % 1,3 butadiene, 560 parts per million (ppm) ethyl acetylene with the balance being butane saturates, all wt. % based on the total weight of the C₄ stream. This C₄ stream was fed at a rate of 63 grams per hour (gms/hr) to a reactor loaded with 30 gms (dry resin weight) of a macroporous ion exchange resin made up of sulfonated polystyrene resins crosslinked by divinylbenzene. The resin contained approximately 5.2 milliequivalents of acid sites per dry gram of resin. The reactor inlet temperature was controlled at 185° F. in order to maintain conversion of isobutylene in the range of 60-70%.

Measurements of bed pressure drop, made through a differential pressure transducer, showed the following build-up with time over the 6 inches of catalyst bed:

| Time of Operation, hours | Measured Pressure Drop, psi |
|---|---|
| 0 | 0.22 |
| 2,000 | 0.35 |
| 3,000 | 0.80 |
| 3,400 | 1.50 |

At this point the run was terminated and the catalyst was removed from the reactor with great difficulty due to the manner in which the particles were packed together. The catalyst could be removed only after the reactor tube was cut along its longitudinal axial direction.

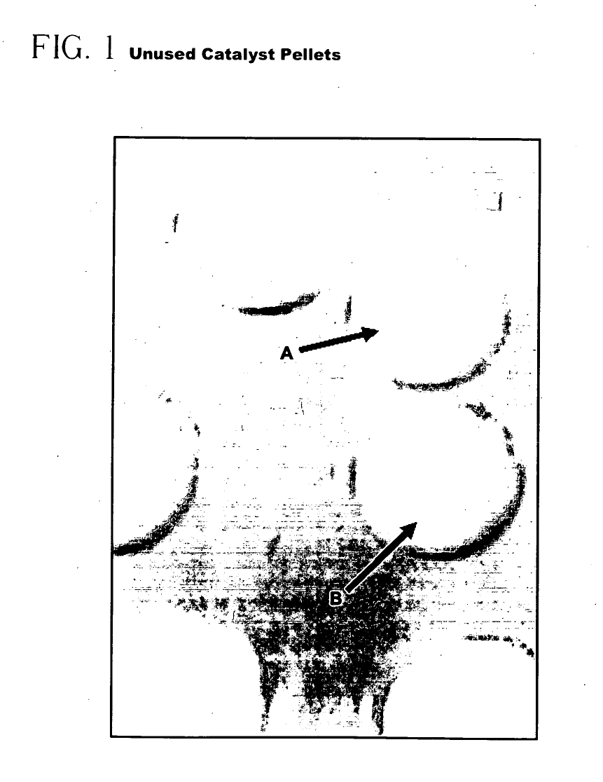

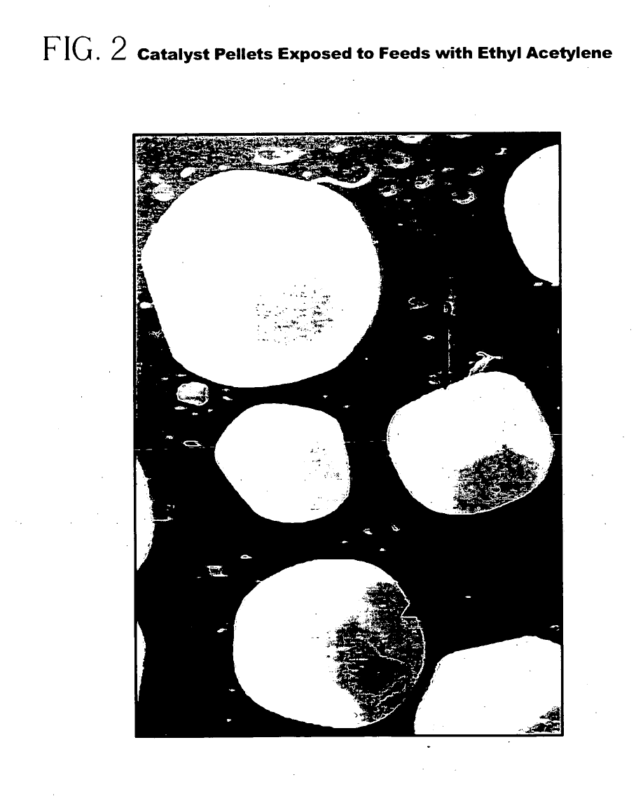

The catalyst was examined microscopically. FIG. 2 is an image of several individual resin pellets, representative of the remainder of the pellets removed from the reactor. FIG. 1 shows an image of an unused sample of sulfonic acid resin pellets. The individual pellets are spherical in shape. Two individual pellets are highlighted by the callouts A and B. FIG. 2 shows that the used resin pellets from this Example no longer retain the spherical shape of the original pellets. Callouts C and D highlight the flat surfaces of the individual pellets. The flat surfaces formed as the individual pellets were fouled and, therefore, swelled and filled the void volume between the individual pellets. As the pellets grew and were pressed against one another, flat surfaces developed. As the inter-particle void space within the reactor was filled, the pressure drop across the bed increased, eventually causing the termination of the run.

This Example 1 demonstrated that mixed C₄ streams containing ethyl acetylene will cause catalyst fouling and, in turn, cause swelling and reactor plugging problems in the catalyst bed.

### EXAMPLE 2

A sample of a mixed C₄ stream (Raff-1) has a composition of 39 wt. % Isobutylene, 38 wt. % normal butenes, 21 wt. % butanes, 0.9 wt. % 1,3 butadiene and 0.6 wt. % ethyl acetylene, all wt. % based on the total weight of the C₄ stream. This C₄ stream is mixed at a rate of 300 gms/hr with hydrogen at a rate of 2.2 standard liters per hour (slh). The combined mixture is fed to a reactor loaded with 100 gms of selective hydrogenation catalyst, consisting of 0.5 wt. % palladium, based on the total weight of the catalyst, on an alumina support. The reactor is maintained at an average bed temperature of 150° F. and 400 psig. The product for the reactor has a composition of 38 wt. % isobutylene, 33 wt. % normal butenes, 27 wt. % butanes, 0.02 wt. % 1,3 butadiene and no detectable amount of ethyl acetylene, all wt. % based on the total weight of the composition.

This hydrotreated stream is mixed at a rate of 200 gms/hr with tert-butyl alcohol at a rate of 6 gms/hr and sec-butyl alcohol at a rate of 4 gms/hr. This mixture is fed to a reactor containing 100 gms of a macroporous ion exchange resin made up of sulfonated polystyrene resins crosslinked with divinylbenzene containing approximately 5.2 milliequivalents of acid sites per gram of resin. The inlet of the reactor is controlled at 160° F. After accumulating product for several hours, 600 grams per hour of the product is recycled to the inlet of the reactor. Periodically the inlet reactor temperature is raised to maintain conversion levels as the catalyst slowly deactivates.

A typical product distribution is of 12 wt. % isobutylene, 34 wt. % normal butenes, 30 wt. % butanes 0.01 wt % 1,3 butadiene, no detectable ethyl acetylene, 2 wt % t-butyl alcohol, 1 wt % s-butyl alcohol, 17 wt. % C₈ olefin (dimer product), 1 wt. % C₈ ether and 1 wt. % C₁₂ olefin (trimer product), all wt. % based on the total weight of the product.

After operation for periods of time similar to those in Example 1, there is no sign of catalyst swelling as is indicated by no change in the pressure drop across the catalyst bed.

### EXAMPLE 3

A sample of a mixed C₄ stream (Raff-1) was employed that had a composition of 39 wt. % Isobutylene, 38 wt. % normal butenes, 21 wt. % butanes, 0.9 wt. % 1,3 butadiene and no detectable ethyl acetylene, all wt. % based on the total weight of the C₄ stream. This C₄ stream was mixed at a rate of 300 gms/hr with hydrogen at a rate of 2.2 standard liters per hour (slh). The combined mixture was fed to a reactor loaded with 100 grams of selective hydrogenation catalyst, consisting of 0.5 wt. % palladium, based on the total weight of the catalyst, on an alumina support. The reactor was maintained at an average bed temperature of 150° F. and 400 psig. The product of the reactor had a composition of 38 wt. % isobutylene, 33 wt. % normal butenes, 27 wt. % butanes, 0.02 wt. % 1,3 butadiene and no detectable amount of ethyl acetylene, all wt. % based on the total weight of the product.

This hydrotreated stream was mixed at a rate of 200 gms/hr with tert-butyl alcohol at a rate of 6 gms/hr and sec-butyl alcohol at a rate of 4 gms/hr. This mixture was fed to a reactor containing 100 gms of a macroporous ion exchange resin made up of sulfonated polystyrene resins crosslinked with divinylbenzene containing approximately 5.2 milliequivalents of acid sites per gram of resin. The inlet of the reactor was controlled at 160° F. After accumulating product for several hours, 600 gms/hr of the product was recycled to the inlet of the reactor. Periodically the inlet reactor temperature was raised to maintain conversion levels as the catalyst slowly deactivated. Over 2,700 hours, the inlet temperature was slowly raised to 175° F. to maintain conversion of the isobutylene.

The product distribution was 12 wt. % isobutylene, 34 wt. % normal butenes, 30 wt. % butanes 0.01 wt. % 1,3 butadiene, no detectable ethyl acetylene, 2 wt. % t-butyl alcohol, 1 wt. % s-butyl alcohol, 17 wt. % C₈ olefin (dimer product), 1 wt. % C₈ ether and 1 wt. % C₁₂ olefin (trimer product).

After 2,700 hours the reaction of the hydrotreated material was terminated. There was no sign of catalyst swelling or reactor plugging. The pressure drop across the catalyst bed remained low and stable throughout the run.

This Example 3 demonstrated that when there were no acetylenics present initially, and hydrotreating was employed, there was no catalyst swelling.

### EXAMPLE 4

A sample of a mixed C₄ stream (Raff-1) had a composition of 48 wt. % isobutylene, 42 wt. % normal butenes, 9 wt. % butanes, 0.3 wt. % 1,3 butadiene and no detectable ethyl acetylene. This stream was mixed at a rate of 200 gms/hr with tert-butyl alcohol at a rate of 6 gms/hr and sec-butyl alcohol at a rate of 4 gms/hr. This mixture was fed to a reactor containing 100 gms of a macroporous ion exchange resin made up of sulfonated polystyrene resins crosslinked with divinylbenzene containing approximately 5.2 milliequivalents of acid sites per gram of resin. The reaction of this Example was conducted with the same load of catalyst as used in Example 3 hereinabove.

A typical product distribution consists of 16 wt. % isobutylene, 40 wt. % normal butenes, 11 wt. % butanes 0.15 wt % 1,3 butadiene, no detectable ethyl acetylene, 2 wt. % t-butyl alcohol, 1 wt. % s-butyl alcohol, 19 wt. % C₈ olefin (dimer product), 1 wt. % C₈ ether and 2 wt. % C₁₂ olefin (trimer product).

After 1,800 hours the reaction of the material was terminated. There was no sign of catalyst swelling or reactor plugging. The pressure drop across the catalyst bed remained low and stable throughout the run.

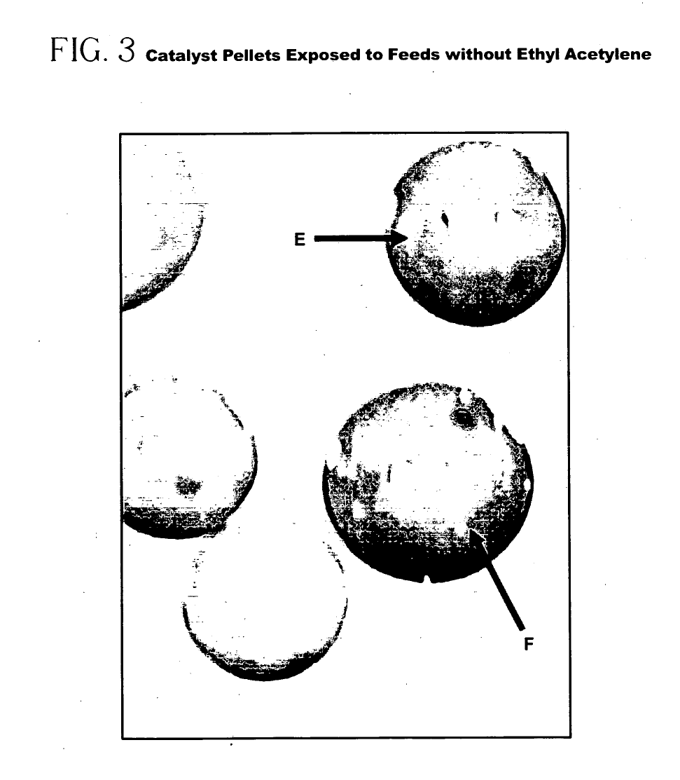

The catalyst was removed from the reactor after 4,500 hours on stream (2,700 hours with hydrotreated feed followed by 1,800 hours with untreated feed containing no ethyl acetylene). FIG. 3 shows an image of the catalyst as removed from the reactor. Callouts E and F highlight the spherical nature of the individual resin pellets. The used resin pellets are in essentially the same shape as the unused resin pellets shown in FIG. 1 and highlighted by callouts A and B. The used resin pellets from Example 4 show none of the distortion that had been present in the resin pellets removed from the reactor in Example 1 and shown in FIG. 2 with distorted shapes of the pellets highlighted by callouts C and D.

This Example 4 demonstrated that when there were no acetylenics present initially and there was no hydrotreating, there was no catalyst swelling.

We claim:

1. A method for catalytically forming diisobutylene from isobutylene comprising providing a feed containing at least in part a mixture of compounds having four carbon atoms per molecule including isobutylene and at least one acetylenic, said feed being deliberately chosen to have an isobutylene content of no more than about 50 weight percent based on the total weight of said feed, at least reducing said acetylenic content of said feed to a level wherein said isobutylene in said feed can be dimerized in the presence of a dimerization catalyst to diisobutylene without unacceptable fouling of said catalyst, and thereafter subjecting said feed to conditions which favor said catalytic dimerization of at least part of said isobutylene content of said feed to diisobutylene.

2. The method of claim 1 wherein said feed contains less than about 60 weight percent isobutylene and less than about 1 weight percent acetylenics, all weight percents based on the total weight of said feed, and said acetylenics are essentially completely removed from said feed.

3. The method of claim 2 wherein said acetylenics are removed by at least one of selective hydrogenation of same to its corresponding olefin, and oligomerization of same to at least one heavier compound.

4. The method of claim 1 wherein said isobutylene dimerization conditions include a temperature of from about 150 to about 250° F., a pressure of from about 250 to about 400 psig, a weight hourly space velocity of fresh feed of from about 0.5 to about 10 reciprocal hours, and at least one catalyst selected from the group consisting of macroporous ion exchange resin made up of sulfonated polystyrene resins crosslinked with divinylbenzene.

5. The method of claim 1 wherein said feed contains from about 30 to about 40 weight percent 1-butene, from about 20 to about 30 weight percent 2-butenes, from about 30 to about 50 weight percent isobutylene, from about 3 to about 7 weight percent n-butane, from about 1 to about 5 weight percent isobutane, up to about 1 weight percent of at least one diolefin, and from about to about 6,000 parts per million of at least one of vinylacetylene and acetylene.
